(19) United States
(12) Patent Application Publication
(10) Pub. No.: US 2021/0202097 A1
(43) Pub. Date: Jul. 1, 2021

(54) SYSTEMS AND METHODS FOR DETERMINING GESTATIONAL DIABETES MELLITUS RISK AND ASSIGNING WORKFLOWS

(71) Applicant: CERNER INNOVATION, INC., Kansas City, KS (US)

(72) Inventors: Deepak Gupta, Bangalore (IN); Kavya Gupta, Bangalore (IN); Harshagiri Ramaprasanna Kumar, Bengaluru (IN); Bibimariyambi Nadaf, Bangalore (IN)

(21) Appl. No.: 17/136,372

(22) Filed: Dec. 29, 2020

Related U.S. Application Data
(60) Provisional application No. 62/955,096, filed on Dec. 30, 2019.

(51) Int. Cl. G16H 50/30; G16H 10/60; A61B 5/145; A61B 5/00

(52) U.S. Cl. CPC G16H 50/30; A61B 5/7275; A61B 5/14532; G16H 10/60

(57) ABSTRACT

Computerized systems and methods are provided for determining the risk of developing gestation diabetes mellitus (GDM) and assigning workflows based on such a determination. The systems and methods can include receiving medical information associated with an individual, determining whether the individual is at risk of developing GDM based on the received medical information, and performing one or more response actions. The one or more response actions can include assigning a workflow for preventative treatment of GDM, providing a notification that the individual is at risk of GDM, or a combination thereof.

### CROSS-REFERENCE TO RELATED APPLICATIONS

This application claims priority to U.S. Provisional Application No.: 62/955,096, filed on Dec. 30, 2019, entitled: “Systems and Methods for Determining Gestational Diabetes Mellitus Risk and Assigning Workflows,” the entire contents of which are incorporated by reference herein.

### BACKGROUND

Gestational diabetes mellitus (GDM) is a known medical complication in pregnancy, where the mother exhibits higher than normal blood glucose levels. GDM may have adverse health effects for both mothers and neonate when the blood glucose level is not controlled. Therefore, there is a need to develop new systems and processes for identifying mothers at risk for GDM so preventative efforts can be taken as early as possible.

### BRIEF DESCRIPTION OF THE SEVERAL VIEWS OF THE DRAWINGS

The present invention is described in detail below with reference to the attached drawing figures, wherein:

FIG. 1 is a block diagram of an exemplary system architecture in which embodiments of the invention may be employed, in accordance with aspects herein;

FIG. 2 is an exemplary system architecture for assessing risk of GDM and assigning workflows, in accordance with aspects herein;

FIG. 3 is a flow diagram of an example method for assessing risk of GDM and assigning workflows, in accordance with aspects herein;

FIGS. 4A and 4B depict a schematic representation of a graphical user interface, in accordance with aspects herein;

FIG. 5 depicts a flow diagram for a method of determining the risk of developing GDM and assigning a workflow, in accordance with aspects herein; and

FIG. 6 depicts a flow diagram for a method of determining the risk of developing GDM and providing a notification of the determined risk, in accordance with aspects herein.

### DETAILED DESCRIPTION

The description of various systems and processes herein is provided to meet statutory requirements. However, the description itself is not intended to limit the scope of the claims. Rather, it is contemplated that the claimed subject matter might also be embodied in other ways, to include different steps or combinations of steps similar to the ones described in this document, in conjunction with other present or future technologies. Moreover, although the terms “step” and/or “block” may be used herein to connote different components of methods employed, the terms should not be interpreted as implying any particular order among or between various steps herein disclosed unless and except when the order of individual steps is explicitly described.

Aspects of the present disclosure relate to systems and methods for determining the risk of developing GDM. In various aspects, the systems and methods disclosed herein can include performing one or more response actions based on a determination of the risk of GDM, such as assigning or providing a GDM-based workflow, providing a notification of the determined risk of GDM, or a combination thereof.

GDM is a known, common medical complication in pregnancy. As discussed above, GDM includes uncontrolled, or abnormally high, blood glucose levels in the pregnant mother, which may lead to medical complications for the mother and/or neonate. Given the potential for adverse effects for the mother and/or the neonate, conventional screening for GDM occurs at about 24-28 weeks of pregnancy by checking the mother's glucose levels, such as with an oral glucose tolerance test. However, 24-28 weeks of pregnancy is late in the second trimester, bridging into the start of the third trimester, and while counseling and treatments may aid in controlling the blood glucose level, it is desirable to begin treatment for GDM, or preventative treatment for a GDM risk, as early as possible. Further, the conventional processes do not provide reliable identification of the risk of GDM early on in pregnancy, e.g., earlier than 24 weeks.

The systems and methods described herein can alleviate one or more of the problems mentioned above. For instance, the systems and methods described herein can determine or assess if an individual is at risk of developing GDM earlier than is performed in conventional systems and processes, e.g., before 24 weeks of pregnancy. In various aspects, the systems and methods disclosed herein can include determining or assessing if an individual is at risk of GDM during the current pregnancy. In such aspects, this assessment or determination can be performed based on medical information associated with the individual. In certain aspects, some or all of the medical information can be obtained at an initial pregnancy medical appointment, or at a pregnancy medical appoint prior to 24 weeks of pregnancy. In various aspects, the systems and methods disclosed herein can make such an assessment of the risk of developing GDM based on medical information that includes age, body mass index, heart or pulse rate, non-invasive systolic blood pressure, non-invasive diastolic blood pressure, estimated creatinine clearance, ethnicity, family medical history, medical history of the individual associated with a prior pregnancy, including prior GDM diagnosis, birth of prior child having macrosomia, blood cortisol level, or a combination thereof. In various aspects, once such medical information is entered into an electronic health record, the systems and methods disclosed herein can automatically make a determination of whether or not the individual requires preventive care for GDM and/or assess the risk of the individual developing GDM based on such medical information. In such aspects, the systems and methods disclosed herein may initiate one or more response actions, such as assigning a workflow for preventative treatment of GDM in an electronic health record and/or transmitting an electronic notification in an electronic health record that the individual is at risk of developing GDM. In such aspects, the one or more response actions can be performed automatically by the system and methods disclosed herein. In various aspects, the determination of GDM risk in response to the individual's medical information being provided into the electronic health record, the adjustment or assigning of a GDM workflow, and/or the notification of the risk of GDM in the individual in the systems and methods described herein can free up processing power and memory for other operations in one or more other medical applications. Additionally, in various aspects, as discussed above, the GDM risk may be assessed or determined early on in an individual's pregnancy, allowing for preventative actions to be taken early on in pregnancy, which increases the chances for preventing the development of GDM. Furthermore, preventing GDM from developing can reduce overall medical costs associated with a pregnancy and delivery, where it is estimated that complications associated with GDM in delivery can increase medical costs at least two-fold, relative to a normal non-GDM delivery.

In certain aspects, as discussed further below, the systems and methods disclosed herein may utilize machine learning to develop one or more risk assessment models to aid in determining the GDM risk. In such aspects, the machine learning service can continually be trained on prior, completed pregnancies, e.g., by utilizing the medical information described above. In one aspect, the machine learning services described herein can be associated with software utilized for managing electronic health records, and may assess the risk of an individual upon entry of the individual's medical information, e.g., the medical information described above.

Accordingly, in one aspect a computerized system is provided. The computerized system can include one or more processors and non-transitory computer storage media storing computer-useable instructions. The computer-useable instructions, when used by the one or more processors, cause the one or more processors to receive medical information associated with an individual, where the individual is less than 24 weeks into a pregnancy; and based on the received medical information, determine that the individual requires preventative treatment for gestational diabetes mellitus (GDM). The computer usable instructions, when used by the one or more processors, further cause the one or more processors to initiate one or more response actions, based on determining that the individual requires preventative treatment for GDM.

In another aspect, a computerized system is provided. The computerized system can include one or more processors and non-transitory computer storage media storing computer-useable instructions. The computer-useable instructions, when used by the one or more processors, cause the one or more processors to receive medical information associated with an individual, where the individual is in the first trimester of pregnancy; and based on the received medical information, determine that the individual is at risk for developing gestational diabetes mellitus (GDM). The computer usable instructions, when used by the one or more processors further cause the one or more processors to automatically transmit an electronic notification that the individual is at risk for developing GDM.

In yet another aspect, a computerized system is provided. The computerized system can include one or more processors and non-transitory computer storage media storing computer-useable instructions. The computer-useable instructions, when used by the one or more processors, cause the one or more processors to receive medical information associated with an individual, where the individual is less than 24 weeks into a pregnancy. The medical information associated with the individual includes age, body mass index, heart or pulse rate, non-invasive systolic blood pressure, non-invasive diastolic blood pressure, estimated creatinine clearance, ethnicity, family medical history, medical history of the individual associated with a prior pregnancy, including prior gestational diabetes mellitus (GDM) diagnosis, birth of prior child having macrosomia, blood cortisol level, or a combination thereof. At least a portion of the medical information can be obtained from the individual at an initial pregnancy medical appointment. The computer usable instructions, when used by the one or more processors further cause the one or more processors to, based on the received medical information, determine that the individual requires preventative treatment for GDM. Based on determining that the individual requires preventative treatment for GDM, the computer usable instructions, when used by the one or more processors further cause the one or more processors to initiate one or more response actions. The one or more response actions can include transmitting an electronic signal to assign a workflow for preventative treatment of GDM in an electronic health record associated with the individual, transmitting an electronic notification in an electronic health record associated with the individual that the individual is at risk of GDM, or a combination thereof.

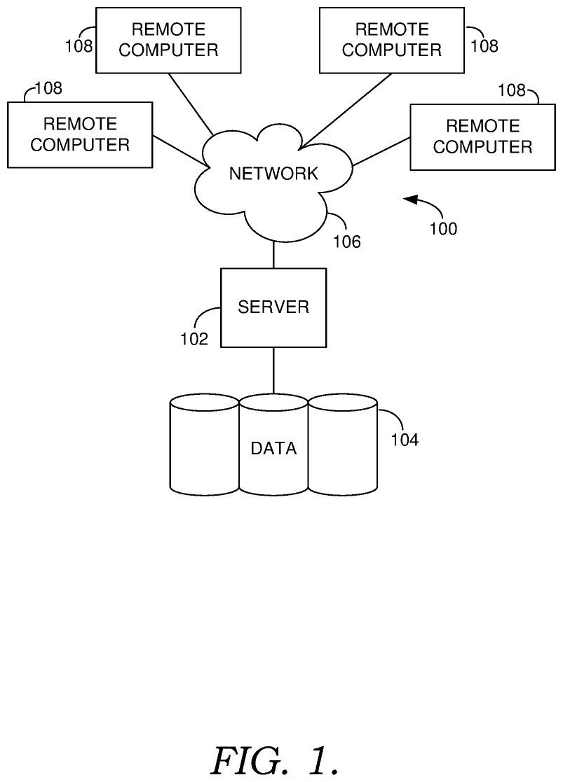

Turning now to the figures, and to FIG. 1 in particular, an example computing environment suitable for use in implementing embodiments of the present invention is shown. FIG. 1 is an example computing environment (e.g., health-information computing-system environment) with which embodiments of the present invention may be implemented. The computing environment is illustrated and designated generally as reference numeral 100. The computing environment 100 is merely an example of one suitable computing environment and is not intended to suggest any limitation as to the scope of use or functionality of the invention. Neither should the computing environment 100 be interpreted as having any dependency or requirement relating to any single component or combination of components illustrated therein. It will be appreciated by those having ordinary skill in the art that the connections illustrated in FIG. 1 are also exemplary as other methods, hardware, software, and devices for establishing a communications link between the components, devices, systems, and entities, as shown in FIG. 1, may be utilized in the implementation of the systems and methods disclosed herein. Although the connections are depicted using one or more solid lines, it will be understood by those having ordinary skill in the art that the exemplary connections of FIG. 1 may be hardwired or wireless, and may use intermediary components that have been omitted or not included in FIG. 1 for simplicity's sake. As such, the absence of components from FIG. 1 should not be interpreted as limiting the systems and methods disclosed herein to exclude additional components and combination(s) of components. Moreover, though devices and components are represented in FIG. 1 as singular devices and components, it will be appreciated that some embodiments may include a plurality of the devices and components such that FIG. 1 should not be considered as limiting the number of a device or component.

The present technology might be operational with numerous other special-purpose computing system environments or configurations. Examples of well-known computing systems, environments, and/or configurations that might be suitable for use with the present invention include personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

The systems and methods disclosed herein may be operational and/or implemented across computing system environments such as a distributed or wireless “cloud” system. Cloud-based computing systems include a model of networked enterprise storage where data is stored in virtualized storage pools. The cloud-based networked enterprise storage may be public, private, or hosted by a third party, in embodiments. In some embodiments, computer programs or software (e.g., applications) are stored in the cloud and executed in the cloud. Generally, computing devices may access the cloud over a wireless network and any information stored in the cloud or computer programs run from the cloud. Accordingly, a cloud-based computing system may be distributed across multiple physical locations.

The present technology might be described in the context of computer-executable instructions, such as program modules, being executed by a computer. Exemplary program modules comprise routines, programs, objects, components, and data structures that perform particular tasks or implement particular abstract data types. The present invention might be practiced in distributed computing environments where tasks are performed by remote processing devices that are linked through a communications network. In a distributed computing environment, program modules might be located in association with local and/or remote computer storage media (e.g., memory storage devices).

With continued reference to FIG. 1, the computing environment 100 comprises a computing device in the form of a control server 102. Exemplary components of the control server 102 comprise a processing unit, internal system memory, and a suitable system bus for coupling various system components, including data store 104, with the control server 102. The system bus might be any of several types of bus structures, including a memory bus or memory controller, a peripheral bus, and a local bus, using any of a variety of bus architectures. Exemplary architectures comprise Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The control server 102 typically includes therein, or has access to, a variety of non-transitory computer-readable media. Computer-readable media can be any available media that might be accessed by control server 102, and includes volatile and nonvolatile media, as well as, removable and nonremovable media. By way of example, and not limitation, computer-readable media may comprise computer storage media and communication media. Computer storage media includes volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by control server 102. Computer-readable media does not include signals per se.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

The control server 102 might operate in a computer network 106 using logical connections to one or more remote computers 108. Remote computers 108 might be located at a variety of locations including operating systems, device drivers and the like. The remote computers might also be physically located in traditional and nontraditional clinical environments so that the entire medical community might be capable of integration on the network. The remote computers might be personal computers, servers, routers, network PCs, peer devices, other common network nodes, or the like and might comprise some or all of the elements described above in relation to the control server. The devices can be personal digital assistants or other like devices. Further, remote computers may be located in a variety of locations including in a medical or research environment, including clinical laboratories (e.g., molecular diagnostic laboratories), hospitals and other individual settings, veterinary environments, ambulatory settings, medical billing and financial offices, hospital administration settings, home medical environments, and clinicians' offices. Medical providers may comprise a treating physician or physicians; specialists such as surgeons, radiologists, cardiologists, and oncologists; emergency medical technicians; physicians' assistants; nurse practitioners; nurses; nurses' aides; pharmacists; dieticians; microbiologists; laboratory experts; laboratory technologists; genetic counselors; researchers; veterinarians; students; and the like. The remote computers 108 might also be physically located in nontraditional clinical environments so that the entire medical community might be capable of integration on the network. The remote computers 108 might be personal computers, servers, routers, network PCs, peer devices, other common network nodes, or the like and might comprise some or all of the elements described above in relation to the control server 102. The devices can be personal digital assistants or other like devices.

Computer networks 106 comprise local area networks (LANs) and/or wide area networks (WANs). Such networking environments are commonplace in offices, enterprise-wide computer networks, intranets, and the Internet. When utilized in a WAN networking environment, the control server 102 might comprise a modem or other means for establishing communications over the WAN, such as the Internet. In a networking environment, program modules or portions thereof might be stored in association with the control server 102, the data store 104, or any of the remote computers 108. For example, various application programs may reside on the memory associated with any one or more of the remote computers 108. It will be appreciated by those of ordinary skill in the art that the network connections shown are exemplary and other means of establishing a communications link between the computers (e.g., control server 102 and remote computers 108) might be utilized.

In operation, an organization might enter commands and information into the control server 102 or convey the commands and information to the control server 102 via one or more of the remote computers 108 through input devices, such as a keyboard, a microphone (e.g., voice inputs), a touch screen, a pointing device (commonly referred to as a mouse), a trackball, or a touch pad. Other input devices comprise satellite dishes, scanners, or the like. Commands and information might also be sent directly from a remote medical device to the control server 102. In addition to a monitor, the control server 102 and/or remote computers 108 might comprise other peripheral output devices, such as speakers and a printer.

Although many other internal components of the control server 102 and the remote computers 108 are not shown, such components and their interconnection are well known. Accordingly, additional details concerning the internal construction of the control server 102 and the remote computers 108 are not further disclosed herein.

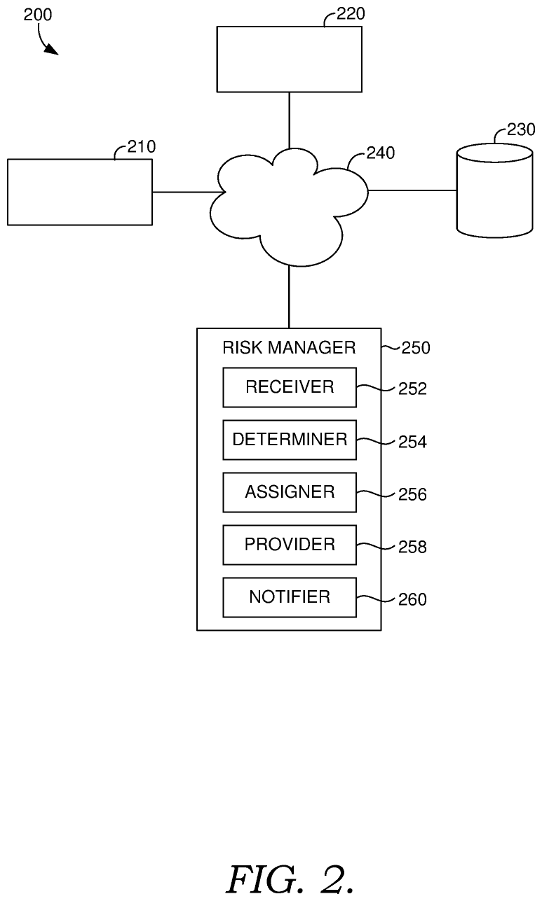

Turning now to FIG. 2, an example system, system 200, for assessing GDM risk and performing one or more response actions is depicted. The system 200 is merely an example of one suitable system and is not intended to suggest any limitation as to the scope of use or functionality of embodiments of the present disclosure. Neither should the system 200 be interpreted as having any dependency or requirement related to any single component or combination of components illustrated herein.

In certain aspects, the system 200 can include the features and properties of one or more of the components of the computing environment 100 of FIG. 1. In the same or alternative aspects, the system 200 can operate and/or function within the computing environment 100 of FIG. 1.

The exemplary system 200 comprises a risk manager 250, a database, 230, a network 240, a computer server 220, and an application 210. As shown, FIG. 2 includes one risk manager 250, one database, 230, one network 240, one computer server 220, and one application 210. However, it is contemplated that the system 200 may comprise more than one of each of these components depending on the needs of the system 200. For example, the system 200 may comprise more than one database 230, which may be located remotely or on the cloud.

In various aspects, the database 230 of FIG. 2 can include medical information associated with one or more electronic medical records. In certain aspects, the medical information can include, but is not limited to, an individual's age, height, weight, body mass index (BMI), heart or pulse rate, non-invasive systolic blood pressure, non-invasive diastolic blood pressure, estimated creatinine clearance, ethnicity, family medical history, medical history of the individual associated with a prior pregnancy, including prior GDM diagnosis, birth of prior child having macrosomia, blood cortisol level, or a combination thereof. In certain aspects, the database 230 can be a database comprised of a plurality of electronic medical records for a plurality of individuals, which can be from one or more medical facilities.

As depicted, the system 200 comprises a risk manager 250. It will be appreciated that some or all of the subcomponents of the risk manager 250 may be accessed via the network 240 and may reside on one or more devices, such as the computer server 220. Additionally, the risk manager 250 may also be integrated into the application 210. It is contemplated that the application 210, in aspects, may be an electronic health record system. Further, in some embodiments, one or more of the illustrated components of the risk manager 250 may be implemented as a stand-alone application. The components described are exemplary in nature and in number and should not be construed as limiting. Any number of components may be employed to achieve the desired functionality within the scope of the embodiments hereof.

In aspects, the risk manager 250 is configured to assess the risk of an individual developing GDM. In certain aspects, at a high level, the risk manager 250 can, among other features, determine or assess if an individual has a risk of developing GDM, based on medical information entered into the individual's electronic health record, and perform one or more response actions, such as assigning a workflow for preventative treatment of GDM and/or providing an electronic notification that the individual is at risk of developing GDM.

As can be seen in the aspect depicted in FIG. 2, the risk manager 250 can include a receiver 252, a determiner 254, an assigner 256, a provider 258, and a notifier 260.

In aspects, the receiver 252 can receive, via the network 240, medical information associated with an individual or an individual's electronic health record, which may be stored in the database 230. In certain aspects, the receiver 252 may be configured to automatically obtain or receive the medical information associated with the individual. For instance, in aspects, once certain medical information is entered into an electronic health record, e.g., via an application 210, such medical information, and optionally additional information associated with the individual, may be transmitted to the risk manager 250, e.g., to the receiver 252. In various aspects, the certain medical information can include an individual's age, height, weight, body mass index (BMI), heart or pulse rate, non-invasive systolic blood pressure, non-invasive diastolic blood pressure, estimated creatinine clearance, ethnicity, family medical history, medical history of the individual associated with a prior pregnancy, including prior GDM diagnosis, birth of prior child having macrosomia, blood cortisol level, or a combination thereof. In one aspect, the certain medical information can consist of an age of the individual, body mass index, heart or pulse rate, non-invasive systolic blood pressure, non-invasive diastolic blood pressure, estimated creatinine clearance, ethnicity, family medical history, medical history of the individual associated with a prior pregnancy including prior GDM diagnosis, birth of prior child having macrosomia, and blood cortisol level of the individual. In another aspect, the certain medical information can consist of an age of the individual, height and weight of the individual, ethnicity, family medical history, medical history of the individual associated with a prior pregnancy, including prior GDM diagnosis, birth of prior child having macrosomia, and blood cortisol level of the individual. In various aspects, the family medical history mentioned above may include, or consist of, family member GDM diagnoses and/or family member diabetes diagnoses. In certain aspects, a null or blank entry for any of the categories of medical information mentioned above may be utilized in the systems and methods disclosed herein. In alternate aspects, the systems and methods disclosed herein may include a preset default value or entry, when in instances, such medical information is not known or available, thereby still allowing for a risk determination to be made.

In certain aspects, the determiner 254 can determine or assess the risk that an individual will develop GDM during a pregnancy based on the medical information received from the receiver 252. In aspects, determining or assessing the risk that an individual will develop GDM during pregnancy can be performed utilizing one or more machine learning models with the medical information discussed above. For instance, in aspects, a classification algorithm can be utilized for determining or assessing a GDM risk, utilizing the medical information for an individual as described above, e.g., comprising or consisting of age of the individual, body mass index, heart or pulse rate, non-invasive systolic blood pressure, non-invasive diastolic blood pressure, estimated creatinine clearance, ethnicity, family medical history, medical history of the individual associated with a prior pregnancy, including prior GDM diagnosis, birth of prior child having macrosomia, and blood cortisol level of the individual. In various aspects, one example, classification algorithm that can be utilized in the systems and methods disclosed herein can include a Random Forest model. It is contemplated that, in alternative aspects, other machine learning methods, such as logistic regression, neural networks, and the like may be used.

In certain aspects, the classification algorithm can be trained utilizing a plurality of electronic health records that include medical information from individuals having completed pregnancies and the results of one or more blood glucose testing during the completed pregnancy. In such aspects, the medical information may include the age of the individual, body mass index, heart or pulse rate, non-invasive systolic blood pressure, non-invasive diastolic blood pressure, estimated creatinine clearance, ethnicity, family medical history, medical history of the individual associated with a prior pregnancy including prior GDM diagnosis, birth of prior child having macrosomia, and blood cortisol level of the individual. In certain aspects, the training set can include electronic health records from one or more medical facilities, which may be stored in the database 230.

In one example, a Random Forest classification algorithm was utilized to classify 1000 instances. Table 1 below lists the results and model parameters utilized.

TABLE 1
Classification Model Classifier Output
Medical
Information

Age, ethnicity,
family history,
prior GDM,
macrosomia,
blood cortisol
level,
class
Bagging with
100 iterations
and base
learner

Summary

| | |
|---|---|
| Correctly | 718 |
| Classified | (71.8%) |
| Instances | |
| Incorrectly | 282 |
| Classified | (28.2%) |
| Instances | |
| Kappa statistic | 0.2558 |
| Mean absolute | 0.3282 |
| error | |
| Root mean | 0.4303 |
| squared error | |
| Relative | 80.8339% |
| absolute error | |
| Root relative | 95.5258% |
| absolute error | |
| Total number | 1000 |
| of instances | |

Detailed
accuracy by
class

| | | FP | | | F- | | ROC | PRC | |
|---|---|---|---|---|---|---|---|---|---|
| | TP Rate | Rate | Precision | Recall | Measure | MCC | Area | Area | Class |
| | 0.392 | 0.153 | 0.502 | 0.392 | 0.440 | 0.259 | 0.730 | 0.480 | 0 |
| | 0.847 | 0.608 | 0.779 | 0.847 | 0.811 | 0.259 | 0.730 | 0.864 | 1 |
| Weighted | 0.718 | 0.479 | 0.701 | 0.718 | 0.706 | 0.259 | 0.730 | 0.755 | |
| Average | | | | | | | | | |

Confusion
Matrix

| | | |
|---|---|---|
| (classified as) | a | b |
| a = 0 | 111 | 172 |
| b = 1 | 110 | 607 |

As can be seen in the classifier output shown in Table 1, the classifier model correctly classified 71.8% of the instances of assessing if an individual is at risk of developing or develops GDM, based on the medical information specified above.

In yet another example, a classifier model, e.g., using a Random Forest classification algorithm, was tested and revealed to have over 95% accuracy with an error rate of just 5.8%. In this example, a classifier model with a model data set of 600,065 was tested on an out of time sample of six months. This model data set included the medical information for an individual as described above, e.g., comprising or consisting of age of the individual, body mass index, heart or pulse rate, non-invasive systolic blood pressure, non-invasive diastolic blood pressure, estimated creatinine clearance, ethnicity, family medical history, medical history of the individual associated with a prior pregnancy, including prior GDM diagnosis, birth of prior child having macrosomia, and blood cortisol level of the individual. The gains table is shown in Table 2a, and the confusion matrix is shown in Table 2b.

TABLE 2a
Gains Table

| | | | | | | | Cum. | % Decile | % Cum. | | % model | |
|---|---|---|---|---|---|---|---|---|---|---|---|---|
| | Decile | pred. | | Cum. | | Cum. | pred. | pred. | Pred. | % Perc | capture | |
| Decile | Size | class | Tier | Size | Good | Good | class | class | class | Good | rate | KS |
| (-inf, | 1494 | 1494 | 1 | 1494 | 0 | 0 | 1494 | 100% | 100% | 0% | 20% | 19.74884 |
| 0.346] | | | | | | | | | | | | |
| (0.346, | 1491 | 1491 | 2 | 2985 | 0 | 0 | 2985 | 100% | 100% | 0% | 39% | 39.45803 |
| 0.391] | | | | | | | | | | | | |
| (0.391, | 1492 | 1492 | 3 | 4477 | 0 | 0 | 4477 | 100% | 100% | 0% | 59% | 59.18044 |
| 0.436] | | | | | | | | | | | | |
| (0.436, | 1505 | 1435 | 4 | 5982 | 70 | 70 | 5912 | 95% | 99% | 1% | 78% | 77.19803 |
| 0.471] | | | | | | | | | | | | |
| (0.471, | 1503 | 1217 | 5 | 7485 | 286 | 356 | 7129 | 81% | 95% | 5% | 94% | 89.39834 |
| 0.509] | | | | | | | | | | | | |
| (0.509, | 1480 | 261 | 6 | 8965 | 1219 | 1575 | 7390 | 18% | 82% | 21% | 98% | 76.28144 |
| 0.516] | | | | | | | | | | | | |
| (0.516, | 1486 | 41 | 7 | 10451 | 1445 | 3020 | 7431 | 3% | 71% | 41% | 98% | 57.18492 |
| 0.524] | | | | | | | | | | | | |
| (0.524, | 1489 | 34 | 8 | 11940 | 1455 | 4475 | 7465 | 2% | 63% | 61% | 99% | 37.85997 |
| 0.537] | | | | | | | | | | | | |
| (0.537, | 1490 | 85 | 9 | 13430 | 1405 | 5880 | 7550 | 6% | 56% | 80% | 100% | 19.8887 |
| 0.556] | | | | | | | | | | | | |
| (0.556, | 1493 | 15 | 10 | 14923 | 1478 | 7358 | 7565 | 1% | 51% | 100% | 100% | 0 |
| inf] | | | | | | | | | | | | |

TABLE 2b
Confusion Matrix
Confusion Matrix

| GDM Status | precision | recall | Data Size |
|---|---|---|---|
| Negative (Good) | 0.94 | 0.95 | 7358 |
| Positive (pred. class) | 0.95 | 0.95 | 7565 |

As can be seen in Tables 2a and 2b, on a test data set of 14,923, at Tier 5, the model captured 95% of the GDM positive cases (7129 captured from 7565 total GDM positive cases in the test data set), with an error rate of just 5.8%. The optimal cutoff was 0.4905 and an area under the curve (AUC) of 98%.

In still another example, a classifier model, e.g., using a Random Forest classification algorithm, was tested and revealed to have over 94% accuracy with an error rate of just 4.6%. In this example, a classifier model with a model data set of 50,052 was tested on an out of time sample of three months. This model data set included the medical information for an individual as described above, e.g., comprising or consisting of age of the individual, body mass index, heart or pulse rate, non-invasive systolic blood pressure, non-invasive diastolic blood pressure, estimated creatinine clearance, ethnicity, family medical history, medical history of the individual associated with a prior pregnancy, including prior GDM diagnosis, birth of prior child having macrosomia, and blood cortisol level of the individual. The gains table is shown in Table 3a, and the confusion matrix is shown in Table 3b.

TABLE 3a
Gains Table

| | | | | | | | Cum. | % Decile | % Cum. | | % model | |
|---|---|---|---|---|---|---|---|---|---|---|---|---|
| | Decile | pred. | | Cum. | | Cum. | pred. | pred. | Pred. | % Perc | capture | |
| Decile | Size | class | Tier | Size | Good | Good | class | class | class | Good | rate | KS |
| (-inf, | 2125 | 2125 | 1 | 2125 | 0 | 0 | 2125 | 100% | 100% | 0% | 22% | 21.6285 |
| 0.197] | | | | | | | | | | | | |
| (0.197, | 2118 | 2113 | 2 | 4243 | 5 | 5 | 4238 | 100% | 100% | 0% | 43% | 43.09053 |
| 0.255] | | | | | | | | | | | | |
| (0.255, | 2088 | 2081 | 3 | 6331 | 7 | 12 | 6319 | 100% | 100% | 0% | 64% | 64.20912 |
| 0.326] | | | | | | | | | | | | |
| (0.326, | 2112 | 2026 | 4 | 8443 | 86 | 98 | 8345 | 96% | 99% | 1% | 85% | 84.06744 |
| 0.428] | | | | | | | | | | | | |
| (0.428, | 2109 | 1080 | 5 | 10552 | 1029 | 1127 | 9425 | 51% | 89% | 10% | 96% | 85.93585 |
| 0.558] | | | | | | | | | | | | |
| (0.558, | 2110 | 140 | 6 | 12662 | 1970 | 3097 | 9565 | 7% | 76% | 27% | 97% | 69.89315 |
| 0.598] | | | | | | | | | | | | |
| (0.598, | 2111 | 83 | 7 | 14773 | 2028 | 5125 | 9648 | 4% | 65% | 45% | 98% | 52.75602 |
| 0.635] | | | | | | | | | | | | |
| (0.635, | 2111 | 59 | 8 | 16884 | 2052 | 7177 | 9707 | 3% | 57% | 64% | 99% | 35.16181 |
| 0.68] | | | | | | | | | | | | |
| (0.68, | 2108 | 70 | 9 | 18992 | 2038 | 9215 | 9777 | 3% | 51% | 82% | 100% | 17.8037 |
| 0.744] | | | | | | | | | | | | |
| (0.744, | 2111 | 48 | 10 | 21103 | 2063 | 11278 | 9825 | 2% | 47% | 100% | 100% | 0 |
| inf] | | | | | | | | | | | | |

TABLE 3b
Confusion Matrix
Confusion Matrix

| GDM Status | precision | recall | Data Size |
|---|---|---|---|
| Negative (Good) | 0.95 | 0.95 | 11278 |
| Positive (pred. class) | 0.94 | 0.95 | 9825 |

As can be seen in Tables 3a and 3b, on a test data set of 21,103, at Tier 5, the model captured 96% of the GDM positive cases (9425 captured from 9825 total GDM positive cases in the test data set), with an error rate of just 4.6%. The optimal cutoff was 0.4676 and an area under the curve (AUC) of 98%.

In the example models described above, the top ten features for determining GDM risk are provided in Table 4.

TABLE 4
Top Ten Features of the Model

| | | Feature |
|---|---|---|
| SL | Influencing Features | importance |
| 1 | Heart/Pulse rate | 0.039905 |
| 2 | Body Mass Index = Weight/(Height)² | 0.034102 |
| 3 | Non-invasive Systolic Blood Pressure | 0.02265 |
| 5 | Sum of age grouped by other columns (e.g., one of age | 0.02042 |
| | group 20-25 years, 25-30 years, 30-35 years, or | |
| | 35-40 years) | |
| 4 | Sum of age grouped by other columns (e.g., one of age | 0.019284 |
| | group 20-25 years, 25-30 years, 30-35 years, or | |
| | 35-40 years) | |
| 6 | Non-invasive Diastolic Blood Pressure | 0.019247 |
| 7 | Skew of Estimated Creatinine Clearance grouped by | 0.018245 |
| | other columns | |
| 8 | Skew of Estimated Creatinine Clearance grouped by | 0.017548 |
| | othe rcolumns | |
| 9 | Sum of age grouped by other columns (e.g., one of age | 0.017084 |
| | group 20-25 years, 25-30 years, 30-35 years, or | |
| | 35-40 years) | |
| 10 | Sum of age grouped by other columns (e.g., one of age | 0.016643 |
| | group 20-25 years, 25-30 years, 30-35 years, or | |
| | 35-40 years | |

In various aspects as discussed above, the determiner 254 can determine whether or not, based on received medical information described above, that an individual is at risk of developing GDM during a current pregnancy. In the same or alternative aspects, the determiner 254 can assess, based on received medical information described above, the risk of an individual developing GDM during a current pregnancy. In such aspects, the determiner 254 may determine or assess that it is more likely than not, e.g., 51% chance or more, that the individual may develop GDM. In various aspects, the determiner 254 may determine, based on received medical information described above, that an individual requires preventative treatment for GDM. In aspects where it is determined that an individual requires preventative treatment for GDM, the determine 254 may also have determined or assessed that the individual is at risk of developing GDM.

In aspects, the assigner 256 may assign a workflow for the individual based on a determination made by the determiner 254. In one aspect, if the determiner 254 determines that an individual requires preventative treatment for GDM, or determines that an individual is at risk of developing GDM, the assigner 256 may assign a preventative GDM treatment workflow for the individual. In aspects, the assigner 265 can associate or assign a particular workflow to a particular individual, e.g., by associating that individual's electronic health record or a portion thereof, with a particular workflow. In alternative aspects, if the determiner 254 determines that an individual is not a risk of developing GDM, the assigner can assign a workflow associated with a normal pregnancy that is not complicated by GDM or other associated complications.

In certain aspects, the provider 258 can provide one or more workflows. In one aspect, the provider 258 can provide and populate in an electronic health record, a workflow that was assigned by the assigner 256. In one aspect, the provider 258 may populate a specific, assigned workflow, such as a preventative GDM workflow, into an individual's electronic health record. In one aspect, the provider 258 may populate an assigned workflow into a software application, such as that represented by the application 210. In one aspect, the provider 258 may populate an assigned workflow into a decision support application, e.g., the PowerChart® software manufactured by Cerner Corporation. In aspects, the provider 258 can automatically provide a workflow into the individual's electronic health record in response to a determination by the determiner 254 and/or in response to a workflow being assigned by the assigner 256.

In various aspects, the notifier 260 can provide a notification, e.g., an electronic notification, that an individual is at risk of developing GDM. In various aspects, the notifier 260 can automatically provide or transmit a notification to a medical professional and/or to an individual's electronic health record, e.g., in response to a determination by the determiner 254 and/or in response to a preventative GDM treatment workflow being assigned by the assigner 256. In certain aspects, the notifier 260 can provide such a notification on the same day as a medical encounter with the individual. In the same or alternative aspects, the notifier 260 can provide such a notification during a medical encounter with the individual. For instance, in various aspects, once certain medical information is entered into the individual's electronic health record and the determiner 254 determines that the individual is or is not at risk of developing GDM, the notifier 260 may provide a notification in the individual's electronic health record during the same appointment in which the certain medical information was entered.

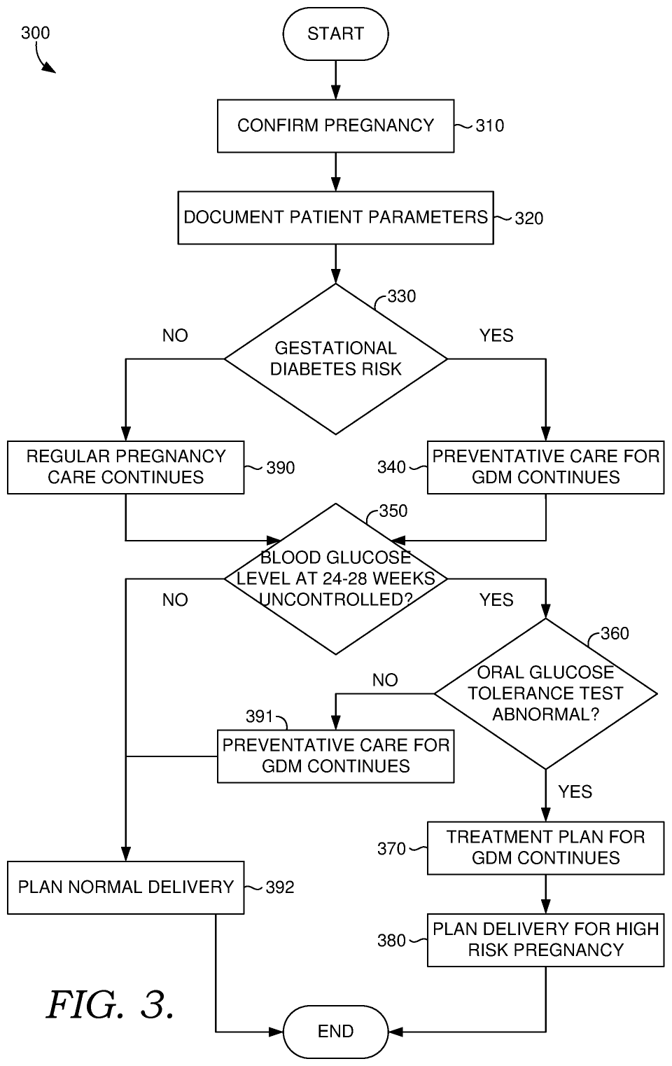

FIG. 3 depicts an example flow chart for one example method 300 for use in certain aspects of the systems described herein. In various aspects, the system 200 of FIG. 2 described above can be utilized to carry out all or a part of the method 300. The method 300 begins with a step 310, where pregnancy is confirmed. In aspects, the step 310 can include receiving information, e.g., pregnancy test results, in an electronic health record of an individual. In the step 320, patient parameters can be documented or received in an electronic health record. In certain aspects, the patient parameters can include the age of the individual, body mass index, heart or pulse rate, non-invasive systolic blood pressure, non-invasive diastolic blood pressure, estimated creatinine clearance, height, weight, ethnicity, family medical history, medical history of the individual associated with a prior pregnancy, including prior GDM diagnosis, birth of prior child having macrosomia, blood cortisol level of the individual, or a combination thereof. In one aspect, the step 320 can occur at substantially the same time or same day as an initial medical encounter with the individual regarding the individual's pregnancy.

In the method 300, at the step 330, it is determined if the individual is at risk of developing GDM. In aspects, the step 330 can include utilizing machine learning, e.g., a classification model, along with the specific medical information obtained at the step 320, to determine if the individual is at risk of developing GDM during the current pregnancy. In aspects, the step 330 can be automatically performed in response to having the specific medical information described above entered into an individual's electronic health record. In certain aspects, the step 330 can be performed before 24 weeks of pregnancy, and/or during the first trimester. In one aspect, the step 330 can occur at substantially the same time or same day as an initial medical encounter with the individual regarding the individual's pregnancy.

In aspects, if at step 330 it is determined that the individual is not at risk, or has a low risk, of developing GDM, then at step 390, the individual may proceed with regular, non-GDM risk pregnancy care. In such aspects, a regular, non-GDM risk workflow can be assigned and/or provided into an individual's electronic health record. In aspects, the regular, non-GDM risk workflow can include, at step 350, a blood glucose test at 24-28 weeks of pregnancy in order to assess whether or not the individual has controlled blood glucose levels. If at step 350, it is determined that the individual has controlled blood glucose level, then at step 392, the individual proceeds with the regular, non-GDM risk workflow that includes the planning of a normal delivery.

In aspects, if at the step 350 it is determined that the individual's blood glucose level is uncontrolled, then at the step 360, the individual will undergo the oral glucose tolerance test discussed further below.

In aspects, if at the step 330 it is determined that the individual is at risk for developing GDM, or at a medium to high risk for developing GDM, it can mean that the individual is more likely than not to develop GDM during the current pregnancy. In such aspects, at step 340, a preventative treatment plan may be pursued. In such aspects, a preventative GDM treatment workflow may be assigned and/or provided in the individual's electronic health record. In aspects, the preventative GDM treatment workflow can include: diet counseling, exercise counseling, blood glucose tests at an increased frequency relative to a non-GDM risk individual, or low GDM risk individual, or a combination thereof. In one aspect, such diet counseling, exercise counseling, and/or blood glucose tests at an increased frequency may commence during the first trimester, or the second trimester.

At the step 350, at 24-28 weeks, the individual who is on a preventative GDM treatment workflow may also undergo a glucose test at 24-28 weeks. If the at step 350, the individual has controlled blood glucose level, e.g., controlled blood glucose level at 24-28 weeks, then the individual who is on a preventative GDM treatment workflow may, at step 392, proceed with a plan for normal delivery. If the at step 350, the individual has an uncontrolled blood glucose level, e.g., an uncontrolled blood glucose level at 24-28 weeks, then the individual who is on a preventative GDM treatment workflow may, at step 360, undergo a conventional oral glucose tolerance test. If at the step 360 it is determined that the individual has a normal oral glucose tolerance test result, then the individual at the step 391 may be assigned a continued preventative GDM workflow, which may include continued diet counseling, exercise counseling, further blood glucose tests, or a combination thereof. In such aspects, where the individual who was on a preventative GDM treatment workflow since the step 340, may have successfully prevented developing GDM. Further, in such aspects, at the step 392, the workflow for such an individual may include a normal delivery plan.

If at the step 360, it is determined that the individual has an abnormal oral glucose tolerance test result, then the individual at the step 370 may be assigned a treatment plan for GDM. In such aspects, the treatment plan may include counseling on various medications including insulin, continued diet counseling, continued exercise counseling, further blood glucose testing at a higher frequency, or a combination thereof. At the step 380, a plan for a high-risk delivery is provided in a workflow for the individual on the treatment plan for GDM at the step 370.

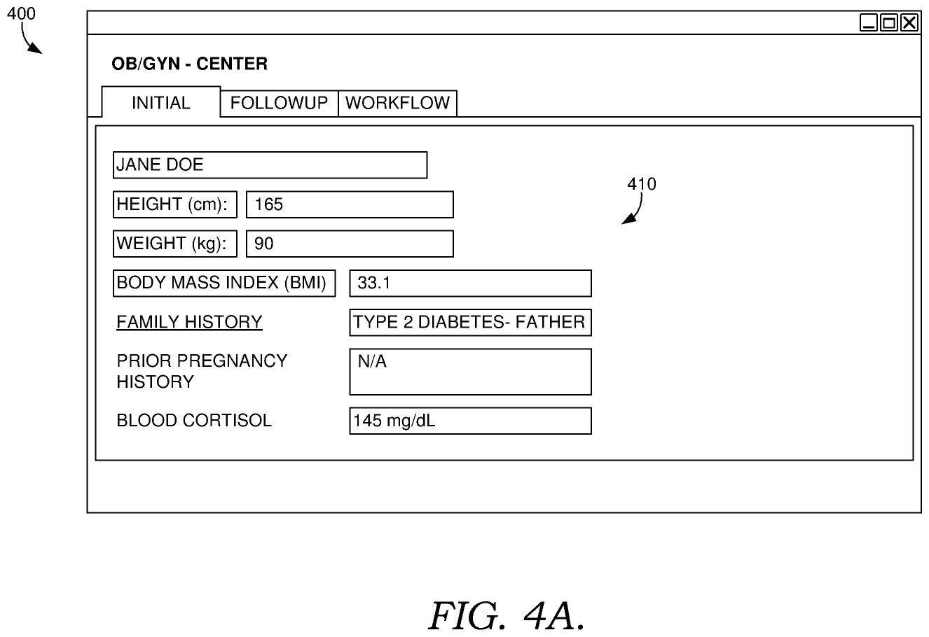

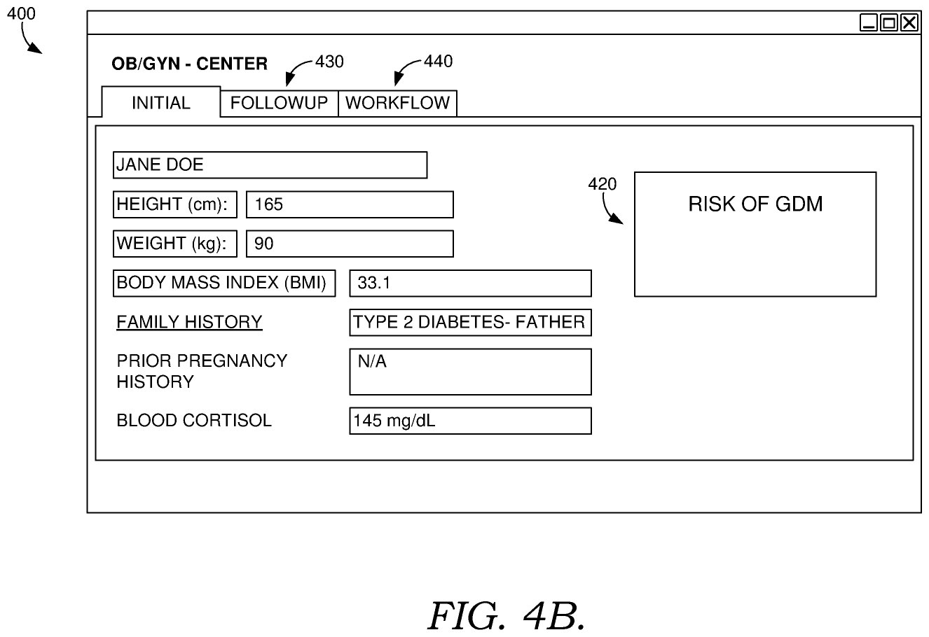

FIG. 4A depicts a graphical user interface 400 that a medical professional may utilize when in a medical encounter with an individual regarding a pregnancy. In one aspect, the graphical user interface 400 can be associated with a decision support application, e.g., the PowerChart® software manufactured by Cerner Corporation. In the aspect depicted in FIG. 4A, the graphical user interface 400 includes user interface elements 410 for entering various medical information. In aspects, such medical information can be the medical information that is utilized by the systems and methods described herein to determine or assess the risk of the individual developing GDM. As discussed in detail above, in certain aspects, the systems and methods disclosed herein can make such a determination or assessment once certain medical information is entered into an electronic health record, e.g., via a graphical user interface 400. Further, as discussed above, in aspects, once such a determination or assessment is made that the individual is at risk of developing GDM, one or more response actions can be performed. For instance, as can be seen in FIG. 4B, the graphical user interface 400 now includes a notification 420 that the individual is at risk of developing GDM. In the same or alternative aspects, the graphical user interface 400 can be populated with the workflow based on the determination or assessment of GDM risk, e.g., via a new interface tab 440 that can display detailed workflow information. Further, in the same or alternative aspects, an additional follow-up tab 430 may also be provided to include any additional follow-up actions necessary during the present visit, such as GDM preventative counseling and/or further testing. While the FIGS. 4A and 4B illustrate one graphical user interface to highlight specific aspects of the present disclosure, it contemplated that alternative or additional visual features may be used to provide the functionality described herein.

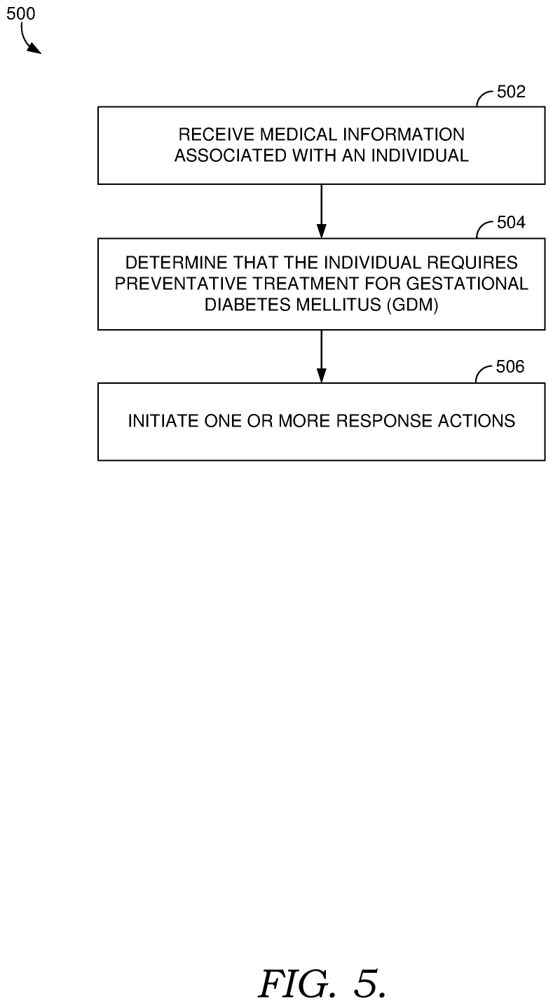

FIG. 5 is a flow diagram of an example method for determining GDM risk and assigning GDM workflows. In step 502 of the method 500, medical information associated with an individual is received. In one aspect, the medical information is received at the receiver 252 of the risk manager 250 of the system 200 of FIG. 2. In aspects, the individual can be at less than 24 weeks of pregnancy when the medical information is received. In various aspects, as discussed above, the medical information associated with the individual comprises or consists of age, body mass index, heart or pulse rate, non-invasive systolic blood pressure, non-invasive diastolic blood pressure, estimated creatinine clearance, ethnicity, family medical history, medical history of the individual associated with a prior pregnancy, including prior GDM diagnosis, birth of prior child having macrosomia, blood cortisol level, or a combination thereof. In certain aspects, at least a portion of the medical information can be obtained from the individual at an initial pregnancy medical appointment.

At the step 504, the method 500 includes determining that an individual requires preventative treatment for GDM. In such aspects, determining that an individual requires preventative treatment for GDM can be based on the medical information that was received in the step 502. In one aspect, a machine learning model is used, e.g., the classification model described above, for determining whether the individual is at risk of developing GDM and/or that the individual requires preventative treatment for GDM. In various aspects, the step 504 can be performed automatically once the medical information is received at the step 502, as discussed above. In aspects, the determiner 254 of the risk manager 250 of the system 200 of FIG. 2 may be utilized to determine that an individual requires preventative treatment for GDM.

At the step 506, the method 500 includes initiating one or more response actions. In such aspects, the one or more response actions can be performed based on the determination made in the step 504. For instance, in one aspect, in response to determining that the individual requires preventative treatment for GDM, one or more response actions may automatically be performed. In various aspects, the one or more response actions can include transmitting a signal to assign a workflow for preventative treatment of GDM in an electronic health record associated with the individual, transmitting an electronic notification in an electronic health record associated with the individual that the individual is at risk of GDM, or a combination thereof. In aspects, the assigner 256, the provider 258, the notifier 260, or a combination thereof, of the risk manager 250 of the system 200 of FIG. 2, may be utilized to perform one or more of the response actions.

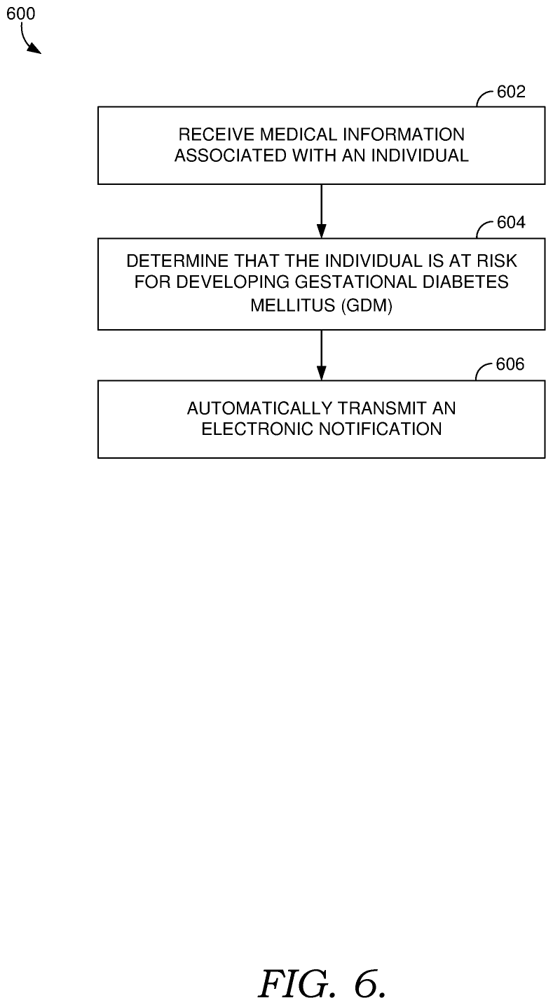

FIG. 6 is a flow diagram of an example method for determining GDM risk. In step 602 of the method 600, medical information associated with an individual is received. In one aspect, the medical information is received at the receiver 252 of the risk manager 250 of the system 200 of FIG. 2. In aspects, the individual can be at less than 24 weeks of pregnancy when the medical information is received. In various aspects, as discussed above, the medical information associated with the individual comprises or consists of age, body mass index, heart or pulse rate, non-invasive systolic blood pressure, non-invasive diastolic blood pressure, estimated creatinine clearance, ethnicity, family medical history, medical history of the individual associated with a prior pregnancy, including prior GDM diagnosis, birth of prior child having macrosomia, blood cortisol level, or a combination thereof. In certain aspects, at least a portion of the medical information can be obtained from the individual at an initial pregnancy medical appointment.

At the step 604, the method 600 includes determining that the individual is at risk for developing GDM. In such aspects, it may be determined that, it is more likely than not, that the individual will develop GDM, based on the medical information that was received in the step 602. In one aspect, machine learning model is used, e.g., the classification model described above, for determining whether the individual is at risk of developing GDM. In various aspects, the step 604 can be performed automatically once the medical information is received at the step 602. In aspects, the determiner 254 of the risk manager 250 of the system 200 of FIG. 2 may be utilized to determine that an individual is at risk for developing GDM.

At the step 606, the method 600 can include automatically transmitting an electronic notification that the individual is at risk of developing GDM. In aspects, as discussed above, the step 606 may happen in response to the determination made in the step 604. In various aspects, the notifier 260 of the risk manager 250 of the system 200 of FIG. 2 may be utilized to transmit and/or provide the notification. In various aspects, the notification may be provided in an electronic health record associated with the individual. In certain aspects, the notification may be transmitted or provided during a medical encounter between the individual and a medical professional.

The present invention has been described in relation to particular embodiments, which are intended in all respects to be illustrative rather than restrictive. Further, the present invention is not limited to these embodiments, but variations and modifications may be made without departing from the scope of the present invention.

What is claimed is:

1. A computerized system comprising:
one or more processors; and
non-transitory computer storage media storing computer-useable instructions that, when used by the one or more processors, cause the one or more processors to:
receive medical information associated with an individual, wherein the individual is less than 24 weeks into a pregnancy;
based on the received medical information, determine that the individual requires preventative treatment for gestational diabetes mellitus (GDM); and
initiate one or more response actions, based on determining that the individual requires preventative treatment for GDM.

2. The computerized system of claim 1, wherein the medical information associated with the individual comprises: an age of the individual, body mass index, heart or pulse rate, non-invasive systolic blood pressure, non-invasive diastolic blood pressure, estimated creatinine clearance, ethnicity, family medical history, medical history of the individual associated with a prior pregnancy, including any prior GDM diagnosis, birth of prior child having macrosomia, blood cortisol level, or a combination thereof.

3. The computerized system of claim 1, wherein the medical information associated with the individual consists of: an age of the individual, body mass index, heart or pulse rate, non-invasive systolic blood pressure, non-invasive diastolic blood pressure, estimated creatinine clearance, ethnicity, family medical history, medical history of the individual associated with a prior pregnancy, including prior GDM diagnosis, birth of prior child having macrosomia, and blood cortisol level.

4. The computerized system of claim 1, wherein a classification model is utilized to determine that the individual requires preventative treatment for gestational diabetes mellitus (GDM).

5. The computerized system of claim 1, wherein the individual is in a first trimester of pregnancy when it is determined that the individual requires preventative treatment for GDM.

6. The computerized system of claim 1, wherein the one or more response actions comprises transmitting a signal to assign a workflow for preventative treatment of GDM in an electronic health record associated with the individual.

7. The computerized system of claim 6, wherein the workflow for preventative treatment of GDM comprises blood glucose monitoring, counseling for dietary modifications, counseling for lifestyle modifications, providing, or a combination thereof.

8. The computerized system of claim 7, wherein the workflow for preventative treatment of GDM comprises blood glucose monitoring beginning in the first trimester of pregnancy.

9. The computerized system of claim 1, wherein the one or more response actions comprises transmitting an electronic notification in an electronic health record associated with the individual that the individual is at risk of GDM.

10. A computerized system comprising:
one or more processors; and
non-transitory computer storage media storing computer-useable instructions that, when used by the one or more processors, cause the one or more processors to:
receive medical information associated with an individual, wherein the individual is in the first trimester of pregnancy;
based on the received medical information, determine that the individual is at risk for developing gestational diabetes mellitus (GDM); and
automatically transmit an electronic notification that the individual is at risk for developing GDM.

11. The computerized system of claim 10, wherein the electronic notification is transmitted to a medical professional on the same day as a medical encounter with the individual.

12. The computerized system of claim 11, wherein the electronic notification is transmitted during a medical encounter with the individual.

13. The computerized system of claim 12, wherein the medical encounter is an initial medical encounter associated with the current pregnancy of the individual.

14. The computerized system of claim 13, wherein the medical information associated with the individual comprises: an age of the individual, body mass index, heart or pulse rate, non-invasive systolic blood pressure, non-invasive diastolic blood pressure, estimated creatinine clearance, ethnicity, family medical history, medical history of the individual associated with a prior pregnancy, including prior GDM diagnosis, birth of prior child having macrosomia, blood cortisol level, or a combination thereof.

15. The computerized system of claim 14, wherein at least a portion of the medical information is obtained from the individual at the initial medical encounter associated with the current pregnancy of the individual.

16. The computerized system of claim 10, wherein the computer-useable instructions further cause the one or more processers to: assign a workflow for preventative treatment of GDM in an electronic health record associated with the individual.

17. The computerized system of claim 16, wherein the workflow for preventative treatment of GDM comprises blood glucose monitoring, counseling for dietary modifications, counseling for lifestyle modifications, or a combination thereof.

18. A computerized system comprising:
one or more processors; and
non-transitory computer storage media storing computer-useable instructions that, when used by the one or more processors, cause the one or more processors to:
receive medical information associated with an individual, wherein the individual is less than 24 weeks into a pregnancy, wherein the medical information associated with the individual comprises an age of the individual, body mass index, heart or pulse rate, non-invasive systolic blood pressure, non-invasive diastolic blood pressure, estimated creatinine clearance, ethnicity, family medical history, medical history of the individual associated with a prior pregnancy, including prior gestational diabetes mellitus (GDM) diagnosis, birth of prior child having macrosomia, blood cortisol level, or a combination thereof, wherein at least a portion of the medical information is obtained from the individual at an initial pregnancy medical appointment;
based on the received medical information, determine that the individual requires preventative treatment for GDM; and
initiate one or more response actions, based on determining that the individual requires preventative treatment for GDM, wherein the one or more response actions comprises: transmitting a signal to assign a workflow for preventative treatment of GDM in an electronic health record associated with the individual, transmitting an electronioc notification in an electronic health record associated with the individual that the individual is at risk of GDM, or a combination thereof.

19. The computerized system of claim 18, wherein the one or more response actions comprises transmitting a signal to assign a workflow for preventative treatment of GDM in an electronic health record associated with the individual, and wherein the workflow for preventative treatment of GDM comprises blood glucose monitoring, counseling for dietary modifications, counseling for lifestyle modifications, or a combination thereof.

20. The computerized system of claim 18, wherein the one or more response actions comprises transmitting an electronic notification in an electronic health record associated with the individual that the individual is at risk of GDM, wherein the electronic notification is transmitted in the electronic health record during the initial pregnancy medical appointment.
